(19) United States
(12) Patent Application Publication
(10) Pub. No.: US 2007/0270768 A1
(43) Pub. Date: Nov. 22, 2007

(54) Mechanical Linkage Mechanism For Ophthalmic Injection Device

(76) Inventors: Bruno Dacquay, Irvine, CA (US); Cesario Dos Santos, Aliso Viejo, CA (US); James Foster, Santa Ana, CA (US); Casey Lind, Irvine, CA (US)

Correspondence Address: ALCON, IP LEGAL, TB4-8, 6201 SOUTH FREEWAY, FORT WORTH, TX, 76134, US

(21) Appl. No.: 11/752,011

(22) Filed: May 22, 2007

Related U.S. Application Data
(63) Continuation-in-part of application No. 60/921,497, filed on Oct. 16, 2006.
Application No. 60/921,497 is a continuation-in-part of application No. 11/435,906, filed on May 17, 2006.

(51) Int. Cl. A61M 5/32

(52) U.S. Cl. 604/272

(57) ABSTRACT

An ophthalmic injection device has a dispensing chamber housing, a plunger, a needle fluidly coupled to a dispensing chamber, a temperature control device, a power source for providing power to the temperature control device, a controller for controlling the temperature control device, and a mechanical linkage mechanism. The interior surface of the dispensing chamber housing partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. A plunger shaft is coupled to the plunger. The temperature control device can alter the temperature of the substance contained therein. The mechanical linkage mechanism has at least two pivots and at least two shafts and transfers force from a lever to the plunger.

### RELATED APPLICATIONS

This application is a continuation-in-part of U.S. patent application Ser. No. 11/581,629 filed Oct. 16, 2006 and U.S. patent application Ser. No. 11/435,906 filed May 17, 2006.

### BACKGROUND OF THE INVENTION

The present invention relates to a single-use medical device and more particularly to an ophthalmic drug delivery device with a mechanical linkage mechanism.

Several diseases and conditions of the posterior segment of the eye threaten vision. Age related macular degeneration (ARMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples.

These, and other diseases, can be treated by injecting a drug into the eye. Such injections are typically manually made using a conventional syringe and needle. FIG. 1 is a perspective view of a prior art syringe used to inject drugs into the eye. In FIG. 1, the syringe includes a needle 105, a luer hub 110, a chamber 115, a plunger 120, a plunger shaft 125, and a thumb rest 130. As is commonly known, the drug to be injected is located in chamber 115. Pushing on the thumb rest 130 causes the plunger 120 to expel the drug through needle 105.

In using such a syringe, the surgeon is required to puncture the eye tissue with the needle, hold the syringe steady, and actuate the syringe plunger (with or without the help of a nurse) to inject the fluid into the eye. The volume injected is typically not controlled in an accurate manner because the vernier on the syringe is not precise relative to the small injection volume. Fluid flow rates are uncontrolled. Reading the vernier is also subject to parallax error. Tissue damage may occur due to an “unsteady” injection. Reflux of the drug may also occur when the needle is removed from the eye.

An effort has been made to control the delivery of small amounts of liquids. A commercially available fluid dispenser is the ULTRA™ positive displacement dispenser available from EFD Inc. of Providence, R.I. The ULTRA dispenser is typically used in the dispensing of small volumes of industrial adhesives. It utilizes a conventional syringe and a custom dispensing tip. The syringe plunger is actuated using an electrical stepper motor and an actuating fluid. Parker Hannifin Corporation of Cleveland, Ohio distributes a small volume liquid dispenser for drug discovery applications made by Aurora Instruments LLC of San Diego, Calif. The Parker/Aurora dispenser utilizes a piezo-electric dispensing mechanism. Ypsomed, Inc. of Switzerland produces a line of injection pens and automated injectors primarily for the self-injection of insulin or hormones by a patient. This product line includes simple disposable pens and electronically-controlled motorized injectors.

U.S. Pat. No. 6,290,690 discloses an ophthalmic system for injecting a viscous fluid (e.g. silicone oil) into the eye while simultaneously aspirating a second viscous fluid (e.g. perflourocarbon liquid) from the eye in a fluid/fluid exchange during surgery to repair a retinal detachment or tear. The system includes a conventional syringe with a plunger. One end of the syringe is fluidly coupled to a source of pneumatic pressure that provides a constant pneumatic pressure to actuate the plunger. The other end of the syringe is fluidly coupled to an infusion cannula via tubing to deliver the viscous fluid to be injected.

It would be desirable to have a portable hand piece for injecting a drug into the eye that includes reliable technology. A mechanical lever can be utilized to provide accurate translation of a plunger to deliver a substance. The lever configuration can be such that it is activated by a finger or thumb to deliver a precise dosage. The hand piece may be a single piece unit or a two-piece device. Placing the more expensive components, including electronics and a battery, in a reusable assembly, while keeping the sterile components in a disposable assembly, improves the efficiency and cost-effectiveness of a drug delivery system. However, a single piece device with a relatively simple structure is also feasible. Such a system provides numerous benefits over prior art injectors.

### SUMMARY OF THE INVENTION

In one embodiment consistent with the principles of the present invention, the present invention is an ophthalmic injection device having a dispensing chamber housing, a plunger, a needle, a temperature control device, a power source for providing power to the temperature control device, a controller for controlling the temperature control device, and a mechanical linkage mechanism. The dispensing chamber housing has an inner surface and an outer surface. The inner surface partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. A plunger shaft is coupled to the plunger. The needle is fluidly coupled to the dispensing chamber. The temperature control device at least partially surrounds the dispensing chamber housing and is capable of altering the temperature of the substance in the dispensing chamber. The mechanical linkage mechanism has at least two pivots and at least two shafts and transfers force from a lever to the plunger.

In another embodiment consistent with the principles of the present invention, the present invention is an ophthalmic injection device having a tip segment attachable to and removable from a limited reuse assembly. The tip segment has a dispensing chamber housing, a plunger, a needle, and a temperature control device. The limited reuse assembly has a power source for providing power to the temperature control device, a controller for controlling the temperature control device, and a mechanical linkage mechanism. The dispensing chamber housing has an inner surface and an outer surface. The inner surface partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. The plunger has a plunger interface for coupling with a plunger shaft. The needle is fluidly coupled to the dispensing chamber. The temperature control device at least partially surrounds the dispensing chamber housing and is capable of altering the temperature of the substance in the dispensing chamber. The mechanical linkage mechanism has at least two pivots and at least two shafts and transfers force from a lever to the plunger.

In another embodiment consistent with the principles of the present invention, the present invention is an ophthalmic injection device having a tip segment attachable to and removable from a limited reuse assembly. The tip segment has a dispensing chamber housing, a plunger, a needle, a temperature control device, and a mechanical linkage mechanism. The dispensing chamber housing has an inner surface and an outer surface. The inner surface partially defines a dispensing chamber for holding a quantity of a substance. The plunger is engaged with the inner surface of the dispensing chamber housing, is capable of sliding in the dispensing chamber housing, and is fluidly sealed to the inner surface of the dispensing chamber housing. The plunger has a plunger interface for coupling with a plunger shaft. The needle is fluidly coupled to the dispensing chamber. The temperature control device at least partially surrounds the dispensing chamber housing and is capable of altering the temperature of the substance in the dispensing chamber. The mechanical linkage mechanism has at least two pivots and at least two shafts and transfers force from a lever to the plunger. The limited reuse assembly has a power source for providing power to the temperature control device and a controller for controlling the temperature control device.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a prior art syringe.

FIG. 2 is one view of an ophthalmic medical device including a disposable tip segment and a limited reuse assembly according to an embodiment of the present invention.

FIG. 3 is a cross section view of a disposable tip segment and a limited reuse assembly according to an embodiment of the present invention.

FIG. 4 is an exploded cross section view of a tip segment for an ophthalmic medical device according to an embodiment of the present invention.

FIG. 5 is a cross section view of an ophthalmic injection device according to the principles of the present invention.

FIG. 6 is a cross section view of a disposable tip segment and a limited reuse assembly according to an embodiment of the present invention.

FIGS. 7A and 7B are cross section views of a mechanical linkage mechanism according to the principles of the present invention.

FIGS. 8A-8C are diagrams showing the trigonometric relationship of a mechanical linkage mechanism according to the principles of the present invention.

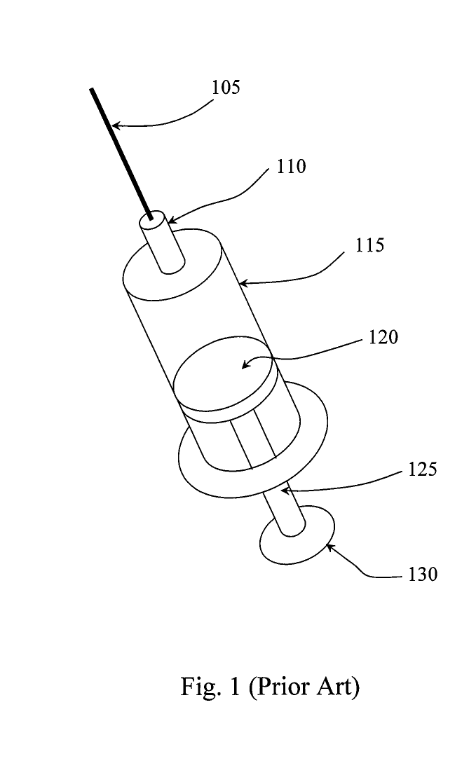

### DETAILED DESCRIPTION OF THE PREFERRED EMBODIMENTS

Reference is now made in detail to the exemplary embodiments of the invention, examples of which are illustrated in the accompanying drawings. Wherever possible, the same reference numbers are used throughout the drawings to refer to the same or like parts.

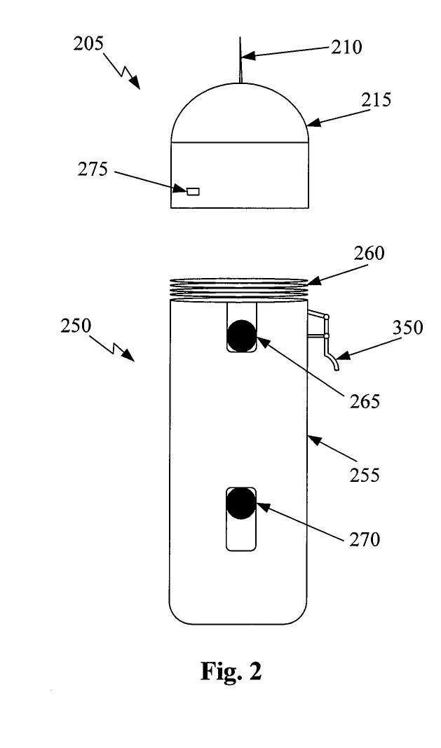

FIG. 2 is one view of an ophthalmic medical device including a disposable tip segment and a limited reuse assembly according to an embodiment of the present invention. In FIG. 2, the medical device includes a tip segment 205 and a limited reuse assembly 250. The tip segment 205 includes a needle 210, a housing 215, and an optional light 275. The limited reuse assembly 250 includes a housing 255, a switch 270, a lock mechanism 265, a lever 350, and a threaded portion 260.

Tip segment 205 is capable of being connected to and removed from limited reuse assembly 250. In this embodiment, tip segment 205 has a threaded portion on an interior surface of housing 215 that screws onto the threaded portion 260 of limited reuse assembly 250. In addition, lock mechanism 265 secures tip segment 215 to limited reuse assembly 250. Lock mechanism 265 may be in the form of a button, a sliding switch, or a cantilevered mechanism. Other mechanisms for connecting tip segment 205 to limited reuse assembly 250, such as those involving structural features that mate with each other, are commonly known in the art and are within the scope of the present invention.

Needle 210 is adapted to deliver a substance, such as a drug, into an eye. Needle 210 may be of any commonly known configuration. Preferably, needle 210 is designed such that its thermal characteristics are conducive to the particular drug delivery application. For example, when a heated drug is to be delivered, needle 210 may be relatively short (several millimeters) in length to facilitate proper delivery of the drug.

Switch 270 is adapted to provide an input to the system. For example, switch 270 may be used to activate the system or to turn on a heater. Other switches, buttons, or user-directed control inputs are commonly known and may be employed with limited reuse assembly 250 and/or tip segment 205.

Optional light 275 is illuminated when tip segment 205 is ready to be used. Optional light 275 may protrude from housing 215, or it may be contained within housing 215, in which case, optional light 275 may be seen through a clear portion of housing 215. In other embodiments, optional light 275 may be replaced by an indicator, such as a liquid crystal display, segmented display, or other device that indicates a status or condition of disposable tip segment 205. For example, optional light 275 may also pulse on and off to indicate other states, such as, but not limited to a system error, fully charged battery, insufficiently charged battery or faulty connection between the tip segment 205 and limited use assembly 250. While shown on tip segment 205, optional light 275 or other indicator may be located on limited reuse assembly 250.

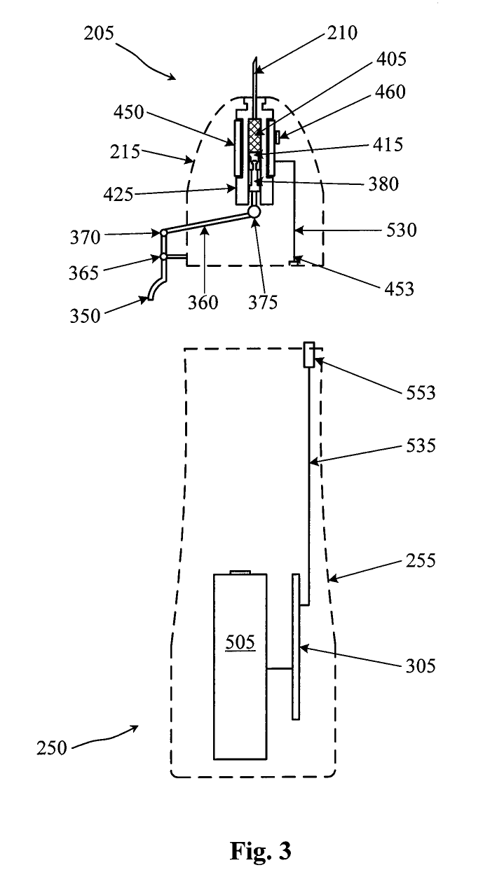

FIG. 3 is cross section view of a disposable tip segment and a limited reuse assembly according to an embodiment of the present invention. FIG. 3 shows how tip segment 205 interfaces with limited reuse assembly 250. In the embodiment of FIG. 3, tip segment 205 includes dispensing chamber housing 425, tip segment housing 215, thermal sensor 460, needle 210, dispensing chamber 405, plunger 415, plunger shaft 380, temperature control device 450, interface 530, tip interface connector 453, and a mechanical linkage mechanism comprising lever 350, pivot 365, shaft 355, coupling 370, shaft 360, and pivot 375. Limited reuse assembly 250 includes power source 505, controller 305, limited reuse assembly housing 255, interface 535, and limited reuse assembly interface connector 553.

In tip segment 205, plunger 415 is adapted to slide within dispensing chamber 405. The outer surface of plunger 415 is fluidly sealed to the inner surface of dispensing chamber housing 425. Dispensing chamber housing 425 surrounds the dispensing chamber 405. Typically, dispensing chamber housing 425 has a cylindrical shape. As such, dispensing chamber 405 also has a cylindrical shape.

Needle 210 is fluidly coupled to dispensing chamber 405. In such a case, a substance contained in dispensing chamber 405 can pass through needle 210 and into an eye. Temperature control device 450 at least partially surrounds dispensing chamber housing 425. In this case, temperature control device 450 is adapted to heat and/or cool dispensing chamber housing 425 and any substance contained in dispensing chamber 405. Interface 530 connects temperature control device 450 with tip interface connector 453.

Optional thermal sensor 460 provides temperature information to assist in controlling the operation of temperature control device 450. Thermal sensor 460 may be located near dispensing chamber housing 425 and measure a temperature near dispensing chamber housing 425 or may be located in thermal contact with dispensing chamber housing 425, in which case it measures a temperature of dispensing chamber housing 425. Thermal sensor 460 may be any of a number of different devices that can provide temperature information. For example, thermal sensor 460 may be a thermocouple or a resistive device whose resistance varies with temperature. Thermal sensor is also electrically coupled to interface 530 or other similar interface.

The components of tip segment 205, including dispensing chamber housing 425, temperature control device 450, and plunger 415 are at least partially enclosed by tip segment housing 215. In one embodiment consistent with the principles of the present invention, plunger 415 is sealed to the interior surface of dispensing chamber housing 425. This seal prevents contamination of any substance contained in dispensing chamber 405. For medical purposes, such a seal is desirable. This seal can be located at any point on plunger 415 or dispensing chamber housing 425.

In limited reuse assembly 250, power source 505 is typically a rechargeable battery, such as a lithium ion battery, although other types of batteries may be employed. In addition, any other type of power cell is appropriate for power source 505. Power source 505 provides current to dispensing chamber housing 425 to heat it and change its shape. Optionally, power source 505 can be removed from housing 255 through a door or other similar feature (not shown).

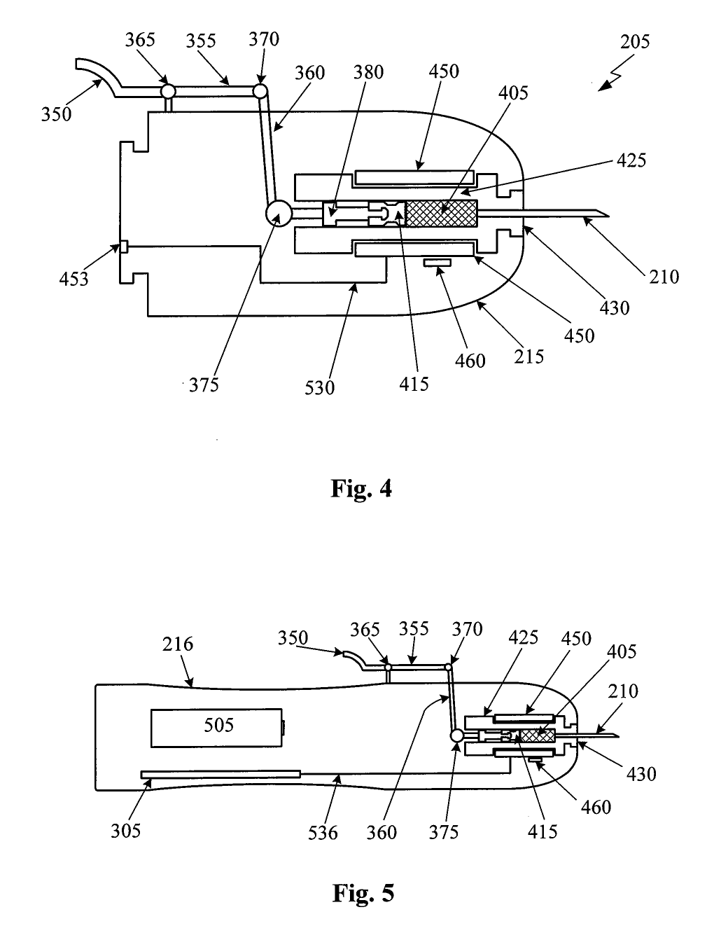

Controller 305 is typically an integrated circuit with power, input, and output pins capable of performing logic functions. In various embodiments, controller 305 is a targeted device controller. In such a case, controller 305 performs specific control functions targeted to a specific device or component, such as a temperature control device or a power supply. For example, a temperature control device controller has the basic functionality to control current delivered to dispensing chamber housing 425. In other embodiments, controller 305 is a microprocessor. In such a case, controller 305 is programmable so that it can function to control more than one component of the device. In other cases, controller 305 is not a programmable microprocessor, but instead is a special purpose controller configured to control different components that perform different functions. While depicted as one component in FIG. 4, controller 305 may be made of many different components or integrated circuits.

Controller 305 is connected via interface 535 to limited reuse assembly interface connecter 553. Limited reuse assembly interface connecter 553 is located on a top surface of limited reuse assembly housing 255. In this manner, limited reuse assembly interface connector 553 is adapted to be connected with tip interface connector 453 to provide an electrical connection between tip segment 205 and limited reuse assembly 250.

An interface between power source 505 and controller 305 allows controller 305 to control operation of power source 505. In such a case, controller 305 may control the charging and the discharging of power source 505 when power source 505 is a rechargeable battery.

In operation, when tip segment 205 is connected to limited reuse assembly 250, the device is ready to be used for an injection. When lever 350 is actuated, coupling 370 moves towards tip segment housing 215. Shaft 360 is rotated moving pivot 375 and plunger 415 upward toward needle 210. A substance located in dispensing chamber 405 is then expelled through needle 210.

Controller 305 controls the operation of temperature control device 450. Temperature control device 450 is adapted to heat and/or cool dispensing chamber housing 425 and its contents. Since dispensing chamber housing 425 is at least partially thermally conductive, heating or cooling dispensing chamber housing 425 heats or cools a substance located in dispensing chamber 405. Temperature information can be transferred from thermal sensor 460 through interface 530, tip interface connector 453, limited reuse assembly interface connector 553, and interface 535 back to controller 305. This temperature information can be used to control the operation of temperature control device 450. When temperature control device 450 is a heater, controller 305 controls the amount of current that is sent to temperature control device 450. The more current sent to temperature control device 450, the hotter it gets. In such a manner, controller 305 can use a feed back loop utilizing information from thermal sensor 460 to control the operation of temperature control device 450. Any suitable type of control algorithm, such as a proportional integral derivative (PID) algorithm, can be used to control the operation of temperature control device 450.

A substance to be delivered into an eye, typically a drug suspended in a phase transition compound, is located in dispensing chamber 405. In this manner, the drug and phase transition compound are contacted by the inner surface of dispensing chamber housing 425. The phase transition compound is in a solid or semi-solid state at lower temperatures and in a more liquid state at higher temperatures. Such a compound can be heated by the application of current to temperature control device 450 to a more liquid state and injected into the eye where it forms a bolus that erodes over time.

In one embodiment of the present invention, the substance located in dispensing chamber 405 is a drug that is preloaded into the dispensing chamber. In such a case, tip segment 205 is appropriate as a single use consumable product. Such a disposable product can be assembled at a factory with a dosage of a drug installed.

FIG. 4 is an exploded cross section view of a tip segment for an ophthalmic medical device according to an embodiment of the present invention. In FIG. 4, tip segment 205 includes dispensing chamber housing 425, tip segment housing 215, thermal sensor 460, needle 210, dispensing chamber 405, plunger 415, plunger shaft 380, temperature control device 450, interface 530, tip interface connector 453, an optional luer 430, and a mechanical linkage mechanism comprising lever 350, pivot 365, shaft 355, coupling 370, shaft 360, and pivot 375. Optional luer secures needle 210 to dispensing chamber housing 425.

In the embodiment of FIG. 4, temperature control device 450 is activated to bring a substance in dispensing chamber 405 to the proper temperature. Thermal sensor 460 provides temperature information to controller 305 (not shown) to control temperature control device 450. After the substance has reached the proper temperature, lever 350 is actuated to drive plunger toward needle 210 to dispense a substance contained in dispensing chamber 405.

The mechanical linkage mechanism that includes lever 350 is designed to use a force applied to lever 350 to translate plunger 415 within dispensing chamber housing 425. Lever 350 is hand actuated with a finger or thumb. In one embodiment, a thumb is used to rotate lever 350 about pivot 365. Since lever 350 is rigidly connected to shaft 355, when lever 350 is rotated upward about pivot 365, shaft 355 rotates downward about pivot 365. This in turn causes coupling 370 to rotate downward about pivot 365. Shaft 360 rotates downward about pivot 375 moving plunger shaft 380 (and plunger 415 to which plunger shaft 380 is rigidly connected) toward needle 210. The movement of plunger 415 dispenses the substance contained in dispensing chamber 405.

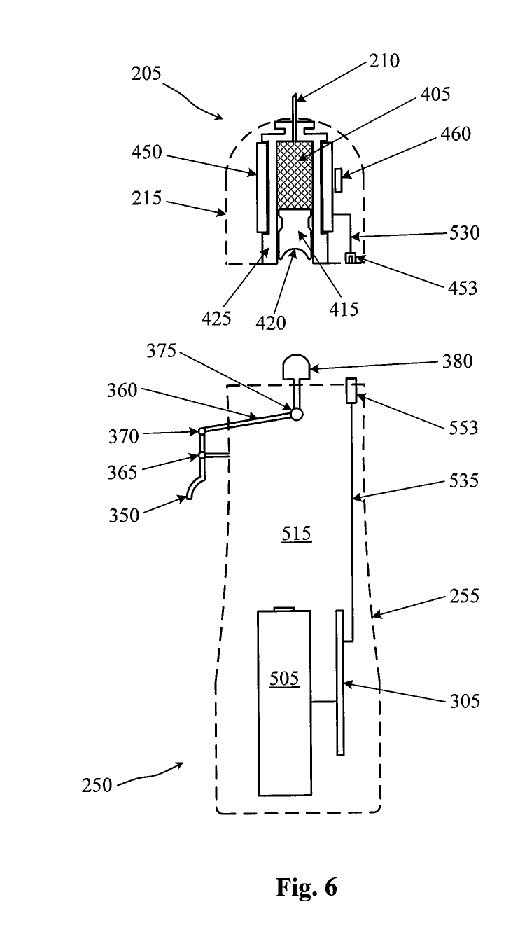

FIG. 5 is a cross section view of an ophthalmic injection device according to the principles of the present invention. In FIG. 5, the injection device is integrated into a single unit. The single piece device of FIG. 6 operates in the same manner as the two piece device previously described. In FIG. 6, the device includes dispensing chamber housing 425, dispensing chamber 405, needle 210, thermal sensor 460, interface 536, controller 305, power source 505, and housing 216. In FIG. 6, a single interface 536 is used instead of two separate interfaces (530 and 535) and two separate connectors (453 and 553). Housing 216 encloses the components pictured.

FIG. 6 is cross section view of a disposable tip segment and a limited reuse assembly according to an embodiment of the present invention. In FIG. 6, the mechanical linkage mechanism is in limited reuse assembly 250 and not in tip segment 205 as depicted in FIG. 3. In FIG. 6, plunger shaft 380 interfaces with plunger 415 at plunger interface 420. Any number of different interfaces can be used including interfaces that are rigidly connected when engaged or those in which force is only transferred in a single direction (as shown in FIG. 6). The embodiment of FIG. 6 has the characteristics and operates in the same manner as the embodiment of FIG. 3.

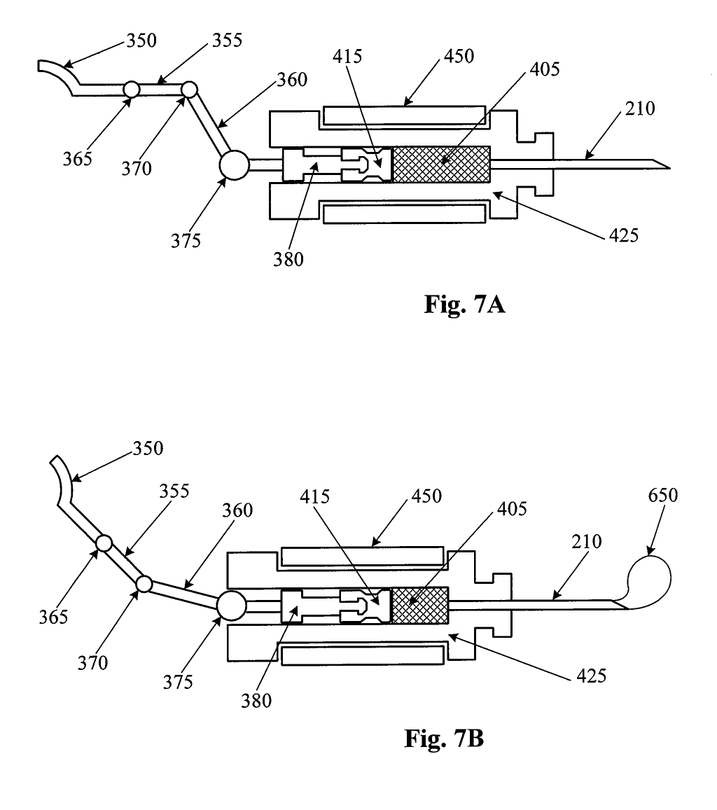

FIGS. 7A and B are cross section views of a mechanical linkage mechanism according to the principles of the present invention. In FIG. 7A, lever 350 has not been actuated. In FIG. 7B, lever 350 has been actuated and a substance 650 has been dispensed from dispensing chamber 405.

In FIGS. 7A and 7B, lever 350 is rigidly connected to shaft 355. Pivot 365 is disposed between lever 350 and shaft 355 so as to provide a first rotation point. Pivot 365 is stationary. A rotatable coupling 370 joins shaft 355 to shaft 360. In this manner, shaft 355 can rotate with respect to shaft 360. Pivot 375 connects shaft 360 to plunger shaft 380. In this manner, shaft 360 rotates about pivot 375. Pivot 375 moves in a direction along plunger shaft 380. Since plunger shaft 380 is constrained to move only in a direction along dispensing chamber housing 425, plunger shaft 380 does not rotate. Instead, when shaft 360 rotates about pivot 375, plunger shaft 380 moves in dispensing chamber housing 425.

When a force is applied to lever 350, lever 350 and shaft 355 rotate about pivot 365. When the force rotates lever 350 upward, shaft 355 is rotated downward. Coupling 370 is moved downward along the arc of a circle with a radius equal to the length of shaft 355 and with its center at pivot 365. As coupling 370 moves, shaft 360 also moves. In this case, shaft 360 moves generally downward and rotates about pivot 375. Pivot 375 moves along an axis defined by plunger shaft 380. As shaft 360 moves downward, pivot 375 moves toward needle 210. Plunger 415 moves in dispensing chamber housing 425 to expel substance 460 as shown in FIG. 6B.

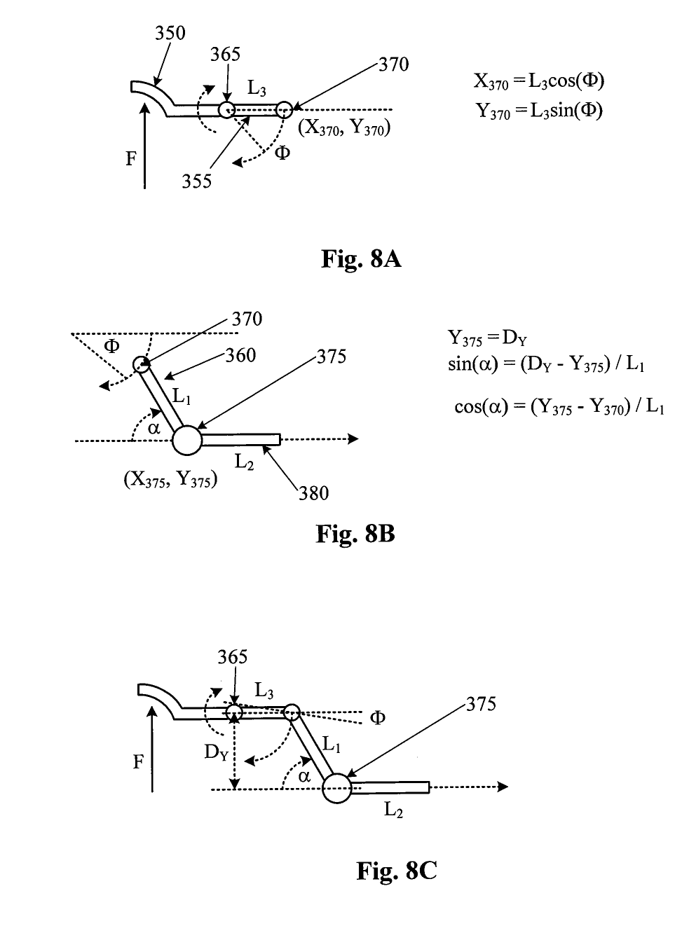

FIGS. 8A-8C are diagrams showing the trigonometric relationship of a mechanical linkage mechanism according to the principles of the present invention. In FIGS. 8A-8C, Φ measures the angle of the arc through which coupling 370 travels. This is also the angle through which lever 350 and shaft 355 rotates. The point (X₃₇₀, Y₃₇₀) is the position of coupling 370. The angle α is the complement of the angle through which shaft 360 travels. In other words, α measures the angle from an axis defined by plunger shaft 380 to shaft 360. L₁ is the length of shaft 360, L₂ is the length of plunger shaft 380, and L₃ is the length of shaft 355. Pivot 365 is stationary and pivot 375 moves only in a direction along the dashed straight arrow. The point (X₃₇₅, Y₃₇₅) is the position of coupling 375. The distance D_{Y} is the distance between pivot 365 and the dashed line along which plunger shaft 380 travels. Given these parameters, a simple trigonometric relationship among the various components is shown.

From the above, it may be appreciated that the present invention provides an improved system for delivering precise volumes of a substance into an eye. The present invention provides a mechanical linkage mechanism that can be easily actuated by the hand to deliver a substance into an eye. In one embodiment, a disposable tip segment that interfaces with a limited reuse assembly is employed. In another embodiment, a single unit is employed. The present invention is illustrated herein by example, and various modifications may be made by a person of ordinary skill in the art.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An ophthalmic injection device comprising:
a dispensing chamber housing having an inner surface and an outer surface, the inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing;
a plunger shaft coupled to the plunger;
a needle fluidly coupled to the dispensing chamber;
a temperature control device at least partially surrounding the dispensing chamber housing, the temperature control device for altering a temperature of the substance in the dispensing chamber;
a power source for providing power to the temperature control device;
a controller for controlling the temperature control device; and
a mechanical linkage mechanism for transferring a force from a lever to the plunger, the mechanical linkage mechanism comprising at least two pivots and at least two shafts.

2. The device of claim 1 further comprising:
a thermal sensor located near the dispensing chamber housing, the thermal sensor for measuring a temperature.

3. The device of claim 2 further comprising:
an interface connecting the thermal sensor to the controller.

4. The device of claim 3 wherein the controller uses the measured temperature to control the temperature control device.

5. The device of claim 1 wherein an end of the lever of the mechanical linkage mechanism terminates at a first pivot such that when the force is applied to the lever, the lever rotates about the first pivot.

6. The device of claim 5 wherein a first end of a first shaft terminates at the first pivot and a second end of the first shaft terminates at a coupling such that when the force is applied to the lever, the first shaft rotates about the first pivot.

7. The device of claim 6 wherein a first end of a second shaft terminates at the coupling and a second end of the second shaft terminates at a second pivot, the second pivot connected to the plunger shaft, such that movement of the second shaft results in movement of the plunger shaft.

8. The device of claim 1 wherein the power source is a rechargeable battery.

9. The device of claim 1 wherein the temperature control device is a heater.

10. The device of claim 1 wherein the substance is a drug for treating a condition of the eye.

11. The device of claim 1 further comprising:
an indicator for providing information about a status of the device.

12. An ophthalmic injection device comprising:
a tip segment attachable to and removable from a limited reuse assembly;
the tip segment comprising:
a dispensing chamber housing having an inner surface and an outer surface, the inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing, the plunger having a plunger interface for coupling with a plunger shaft;
a needle fluidly coupled to the dispensing chamber; and
a temperature control device at least partially surrounding the dispensing chamber housing, the temperature control device for altering a temperature of the substance in the dispensing chamber;
the limited reuse assembly comprising:
a power source for providing power to the temperature control device;
a controller for controlling the temperature control device; and
a mechanical linkage mechanism for transferring a force from a lever to the plunger, the mechanical linkage mechanism comprising at least two pivots and at least two shafts.

13. The device of claim 12 wherein the tip segment further comprises:
a thermal sensor located near the dispensing chamber housing, the thermal sensor for measuring a temperature.

14. The device of claim 13 further comprising:
an interface connecting the thermal sensor to the controller.

15. The device of claim 14 wherein the controller uses the measured temperature to control the temperature control device.

16. The device of claim 12 wherein an end of the lever of the mechanical linkage mechanism terminates at a first pivot such that when the force is applied to the lever, the lever rotates about the first pivot.

17. The device of claim 16 wherein a first end of a first shaft terminates at the first pivot and a second end of the first shaft terminates at a coupling such that when the force is applied to the lever, the first shaft rotates about the first pivot.

18. The device of claim 17 wherein a first end of a second shaft terminates at the coupling and a second end of the second shaft terminates at a second pivot, the second pivot connected to a plunger shaft, such that movement of the second shaft results in movement of the plunger shaft.

19. The device of claim 12 wherein motion is transferred to the plunger from the mechanical linkage mechanism only in a dispensing direction.

20. The device of claim 12 wherein the power source is a rechargeable battery.

21. The device of claim 12 wherein the temperature control device is a heater.

22. The device of claim 12 wherein the substance is a drug for treating a condition of the eye.

23. The device of claim 12 wherein the limited reuse assembly further comprises:
an indicator for providing information about a status of the device.

24. An ophthalmic injection system comprising:
a tip segment attachable to and removable from a limited reuse assembly;
the tip segment comprising:
a dispensing chamber housing having an inner surface and an outer surface, the inner surface partially defining a dispensing chamber for holding a quantity of a substance;
a plunger engaged with the inner surface of the dispensing chamber housing, the plunger capable of sliding in the dispensing chamber housing, the plunger fluidly sealed to the inner surface of the dispensing chamber housing, the plunger having a plunger interface for coupling with a plunger shaft;
a needle fluidly coupled to the dispensing chamber;
a temperature control device at least partially surrounding the dispensing chamber housing, the temperature control device for altering a temperature of the substance in the dispensing chamber; and
a mechanical linkage mechanism for transferring a force from a lever to the plunger, the mechanical linkage mechanism comprising at least two pivots and at least two shafts;
the limited reuse assembly comprising:
a power source for providing power to the temperature control device; and
a controller for controlling the temperature control device.

25. The device of claim 24 wherein the tip segment further comprises:
a thermal sensor located near the dispensing chamber housing, the thermal sensor for measuring a temperature.

26. The device of claim 24 wherein an end of the lever of the mechanical linkage mechanism terminates at a first pivot such that when the force is applied to the lever, the lever rotates about the first pivot.

27. The device of claim 26 wherein a first end of a first shaft terminates at the first pivot and a second end of the first shaft terminates at a coupling such that when the force is applied to the lever, the first shaft rotates about the first pivot.

28. The device of claim 27 wherein a first end of a second shaft terminates at the coupling and a second end of the second shaft terminates at a second pivot, the second pivot connected to a plunger shaft, such that movement of the second shaft results in movement of the plunger shaft.
